(19) United States
(12) Patent Application Publication
(10) Pub. No.: US 2002/0122792 A1
(43) Pub. Date: Sep. 5, 2002

(54) INDUCTION OF NEOANGIOGENESIS IN ISCHEMIC MYOCARDIUM

(76) Inventor: THOMAS J. STEGMANN, PETERSBERG (DE)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/358,780

(22) Filed: Jul. 22, 1999

Related U.S. Application Data
(60) Provisional application No. 60/093,962, filed on Jul. 24, 1998.

(51) Int. Cl. A61K 38/43

(52) U.S. Cl. 424/94.1

(57) ABSTRACT

The present invention relates to the treatment of coronary heart disease by revascularization therapy, and more particularly to the intramyocardial injection of a pharmaceutical composition comprising fibroblast growth factor-1 and a physiologic glue for inducing local neoangiogenesis in ischemic myocardium.

### RELATED APPLICATIONS

This application claims priority under §119(e) to Provisional Application No. 60/093,962, filed on Jul. 24, 1998.

### BACKGROUND OF THE INVENTION

The present invention is related to the treatment of coronary heart disease by revascularization therapy, and more particularly to pharmaceutical compositions containing neoangiogenic compounds, procedures for preparing such compounds, and methods for delivering the pharmaceutical compositions to the ischemic myocardium.

Heart attack, or myocardial infarction, due to coronary heart disease (CHD) is the single leading cause of death in the U.S. according to the American Heart Association. Myocardial infarction occurs when the blood supply to part of the heart muscle, or myocardium, is severely reduced or stopped, thereby depriving the myocardium of oxygen. This oxygen deprivation, or ischemia, occurs when one of the coronary arteries which supply blood to the myocardium is blocked. The blockage, or stenosis, most frequently results from atherosclerosis, a condition associated with the buildup of fatty deposits in the vessel walls. Statistics based upon the National Heart, Lung, and Blood Institute's Atherosclerotic Risk in Communities (ARIC) Study (1987-1994) and the Framingham Heart Study, indicate that the CHD-related mortality rate in the U.S. is one of every 4.8 deaths (481,287 deaths in 1995). Over one million new and recurrent cases of heart attack and almost 14 million victims of myocardial ischemia, angina and other manifestations of CHD (7.1 million men and 6.8 million women) are reported each year. Moreover, as many as 3 to 4 million individuals in the U.S. alone ii may have ischemic episodes (silent ischemia) without knowing it.

Procedures currently available for treating CHD and myocardial ischemia include: 1) coronary artery bypass graft, wherein a segment of a vein is harvested from the patient's leg and grafted in such a manner as to reroute blood around the stenosis; 2) percutaneous transluminal coronary angioplasty, or balloon angioplasty, wherein a catheter having a deflated balloon is passed into the stenosed region of the artery and the balloon is then inflated to widen the vessel lumen; 3) laser angioplasty, wherein a catheter having a laser at its distal tip is used to ablate the atherosclerotic plaque; 4) artherectomy, wherein a high-speed rotating ‘burr’ at the end of a catheter is used to grind away the atherosclerotic plaque; and 5) transmyocardial revascularization, in which a series of channels are cut in the myocardium by laser to allow blood from inside the left ventricle to permeate into the ischemic heart muscle. While variations, combinations and improvements in these basic approaches are constantly being developed, each of these alternative methods have significant disadvantages.

Thoracic surgeons performed approximately 573,000 bypass operations in 1995 in the U.S. alone. While coronary artery bypass has the advantage of creating a new path through which blood may flow freely to the myocardium, often by graft directly from the aorta or internal mammary artery, it also has the major disadvantage of requiring highly invasive open heart surgery. Indeed, the heart is generally stopped in bypass surgery to facilitate anastomosis of the graft to the coronary artery. Oxygenation and circulation are maintained by a heart-lung machine. Consequently, bypass patients face increased risk of damage to the kidneys, brain and other organs. In addition to the medical risks, bypass procedures are very expensive and require significant recovery time. Moreover, for many patients who are at high risk for major invasive surgery or who have advanced stage and/or diffuse CHD, coronary artery bypass procedures are not a viable option. Consequently, these patients must seek alternative treatments.

The most frequently utilized, less invasive alternative to bypass surgery, is percutaneous transluminal coronary angioplasty, commonly referred to as balloon angioplasty. Approximately 434,000 balloon angioplasties were performed in the U.S. in 1995. While such procedures are considerably less invasive and less expensive than coronary bypass surgery, the improvement in blood flow to the myocardium may be small and short-lived. For instance, according to the American Heart Association, an increase in luminal diameter of greater than 20% is considered successful. Furthermore, restenosis occurs within six months in about 25-30% of patients who have undergone successful angioplasty. To reduce the incidence of restenosis following angioplasty, expandable structural supports, referred to as stents, may be deployed during angioplasty to maintain vessel diameter and blood flow. However, the endothelial and smooth muscle cells which comprise the vessel walls tend to infiltrate the stent scaffolding, eventually compromising blood flow. Finally, balloon angioplasty is not recommended for patients with severe diffuse CHD or in patients having greater than 50% occlusion in their left anterior descending (LAD) coronary artery. Thus, balloon angioplasty is neither sufficiently effective nor widely applicable to alleviate the debilitating symptoms of severe myocardial ischemia in many patients.

Two other catheter-based techniques, laser angioplasty and arthrectomy, are directed toward increased blood flow through removal of atherosclerotic plaque. While these techniques may be used alone, they are often used in conjunction with balloon angioplasty to increase luminal diameter. Unfortunately, plaque removed by these methods may generate debris and/or flaps which may cause sudden, dangerous postoperative occlusions.

Transmyocardial revascularization, or laser revascularization, is another procedure, which is both less invasive and less costly than bypass surgery, and has been forwarded as an option for those patients who are at high risk for a second bypass or angioplasty. By providing direct access of the ischemic myocardium to blood within the ventricular chamber, laser revascularization may be useful in treating patients whose coronary artery blockages are too diffuse to be treated effectively with site-directed bypass surgery and/or angioplasty. Unfortunately, the theoretical benefits of laser revascularization have yet to be proven safe and effective over time. Indeed, the generation of an array of channels cut through the walls of the heart by laser vaporization may serve merely as a stop-gap measure to address acute myocardial ischemia, while diminishing the long-term prognosis.

Thus, there remains a substantial gap in treatment options for CHD patients, particularly those who are at high risk for bypass surgery. Indeed, there is a need for a treatment protocol which is less invasive and less expensive than bypass surgery, and more effective than balloon angioplasty and transmyocardial revascularization.

Normal capillaries have a cell population with a low turnover rate of months or years. On occasion, however, a high turnover rate of this cell population is possible even under physiological conditions, and this naturally leads to the rapid growth of new capillaries and other blood vessels. Such a physiological process occurs in the development of the placenta, in fetal growth, and in would healing, as well in the formation of collaterals in response to tissue ischemia. Angiogenic polypeptide growth factors are essential for such processes as capillary growth or neoangiogenesis. These growth factors bring about their effects by significantly increasing cell proliferation, differentiation, and migration via high-affinity receptors on the surfaces of the endothelial cells. Accordingly, the present invention is directed toward revascularization of the myocardium via local-acting, growth factor-stimulated neoangiogenesis.

### SUMMARY OF THE INVENTION

The present invention relates to a method for revascularizing a region of ischemic myocardium in a human heart, which is underperfused as a result of at least one site of coronary artery stenosis. The method comprises the steps of: (1) preparing a pharmaceutical composition comprising fibroblast growth factor-1 (FGF-1) and a physiologic glue, and (2) injecting an amount of the pharmaceutical composition to the ischemic myocardium at or near the at least one site of coronary artery stenosis. The amount of pharmaceutical composition is sufficient to induce local neoangiogenesis.

The FGF-1 is injected at a final concentration in a range of about 0.1 μg/kg body weight per site to about 10 mg/kg body weight per site, and more preferably, at a final concentration in a range of about 10 to 100 μg/kg body weight per site.

The physiologic glue and the FGF-1 are preferably mixed immediately prior to application. In a preferred embodiment of the present method, the physiologic glue is fibrin glue.

The pharmaceutical composition may also include an anticoagulant, such as heparin. The heparin may be applied at a final concentration in a range of about 1 U per ml to about 1000 U per ml.

In one embodiment, the step of injecting the pharmaceutical composition also involves the steps of: (1) making a thoracotomy incision, (2) identifying the at least one site of coronary artery stenosis, (3) administering a β-blocker to reduce the heart rate to a range of about 20-60 beats per minute, and (4) injecting the pharmaceutical composition intramyocardially at or near the at least one site of coronary artery stenosis. The thoracotomy incision may include an anterior left-sided incision, dissecting the costal cartilage over the 5th rib, and opening the left pleural space and the pericardium. In one embodiment, identification of the site(s) of coronary artery stenosis also includes retracting the heart forward using traction sutures.

### BRIEF DESCRIPTION OF THE DRAWINGS

FIG. 1 is an HPLC profile before high purification.

FIG. 2 is an HPLC profile after high purification.

FIG. 3(A) illustrates results of the chorioallantoic membrane assay with application of the growth factor. (B) shows the chorioallantoic membrane assay of the control group. HBGF-I denotes hFGF-1.

FIG. 4(A) is an angiograph showing clearly discernible accumulation of contrast medium at the site of injection in ischemic rat heart. (B) shows no discernible accumulation of contrast medium in the control group. HBGF-I denotes hFGF-1.

FIG. 5(A) is an angiograph showing a pronounced accumulation of contrast medium compared with the control group after injection of the growth factor into the human heart. (B) shows no increase in the accumulation of contrast medium around the IMA/LAD anastomosis. HBGF-I indicates hFGF-1.

FIG. 6(A) is an angiograph showing collateralization of stenoses (arrow): a diagonal branch occluded just distal to its origin was filled through the newly grown capillaries. (B) shows collateralization of stenoses (arrow) by newly grown capillaries: the peripherally stenosed LAD was filled through these vessels. HBGF-I indicates hFGF-1.

FIG. 7 is a quantitative gray value analysis of contrast medium accumulation in the angiography showing a two-threefold increase in local blood flow at the site of injection. HBGF-I indicates hFGF-1.

### DETAILED DESCRIPTION OF THE PREFERRED EMBODIMENT

Several groups have recently established indications for the effective use of angiogenic growth factors to improve blood flow in the presence of tissue ischemia in animal experiments. Yanagisawa-Miwa et al. (1992) Science 257:1401-1402 demonstrated a significant collateralization together with reduction in the size of the infarct after intracoronary administration of growth factor in rabbits. Baffour et al. (1992) J. Vascul. Surg. 16:181-191 also observed formation of collaterals in ischemic extremities after growth factor administration in animals. Similarly, Albes et al. (1994) Ann. Thorac. Surg. 57:444-449 produced an improvement in the blood flow in ischemic tracheal segments implanted subcutaneously in rabbits by injecting growth factor-enriched fibrin glue locally. Thus, preliminary animal studies suggested that angiogenic factors may be useful in stimulating neoangiogenesis in ischemic tissues.

According to one preferred embodiment of the present invention, myocardial ischemia resulting from one or more predetermined site(s) of coronary artery stenosis is treated by application of an effective amount of a pharmaceutical composition comprising an angiogenic growth factor and a physiologic “glue” at or near the predetermined site(s) of coronary artery stenosis (see Schmacher et al. (1998) Circulation 97:645-650; the disclosure of which is incorporated herein by reference). While the inventor has found that both acidic fibroblast growth factor (FGF-1) and basic fibroblast growth factor (FGF-2) are effective in promoting neoangiogenesis, the acidic form, designated FGF-1, is presently considered the most effective angiogenic growth factor. Notwithstanding the present preference for human FGF-1, this invention encompasses the broader concept and methods of treating CHD in mammals by providing a site-directed injection within the underperfused myocardium, at or near a vessel stenosis, of a pharmaceutical composition comprising any angiogenic substance together with a physiologic glue.

Accordingly, numerous growth factors have been identified which possess significant angiogenic properties, including: both FGF-1 and FGF-2 (FGF is also known as Heparin Binding Growth Factor (HBGF) and Endothelial Cell Growth Factor (ECGF); see e.g. Schlaudraff et al. (1993) Eur. J. Cardio-thorac. Surg. 7:637-644; Fasol et al. (1994) J. Thorac. Cardiovasc. Surg. 107:1432-1439), which are potent mitogens for both vascular endothelial cells as well as the underlying smooth muscle cells; Vascular Endothelial Growth Factor (VEGF), which is mitogenic for the vascular endothelial cells, but not for the underlying smooth muscle cells (see e.g. Isner et al. (1996) Lancet 348:370-374; Dvorak et al. (1991) J. Exp. Med. 174:1275-1278); angiopoietin-1, which mediates the recruitment of smooth muscle cells to the wall of new vessels (Suri et al. (1996) Cell 87:1171-1180); angiopoietin-2, which may prevent smooth muscle cell apposition to the walls of microvessels (Maisonpierre et al. (1997) Science 277:55-60); and Platelet Derived Growth Factor (PDGF), Insulin-Like Growth Factors-I and II (IGF-I and IGF-II), and Transforming Growth Factors-α and β, (TGF-α and TGF-β), and Epidermal Growth Factor, (EGF), all of which have been shown to be potent modulators of endothelial and smooth muscle cell growth.

The final dose of angiogenic factor to be applied at or near each vessel stenosis, is preferably in the range of 0.1 μg/kg body weight per site to about 10 mg/kg body weight per site. More preferably, the final dose of angiogenic factor is within the range of about 1 μg/kg body weight per site to about 1 mg/kg body weight per site. Most preferably, the dose of growth factor is in the range of about 10 to 100 μg/kg body weight per site.

A physiologic glue is included in the pharmaceutical composition in order to enhance the affinity of the growth factor for the ischemic tissue and to prevent the growth factor from rapidly entering the systemic circulation. It is undesirable to have the angiogenic factor enter the systemic circulation for two reasons. First, it would be rapidly diluted to an ineffective concentration, and second, the growth factor may stimulate undesirable growth at sites other than the target site. Preferably, FGF-1 is mixed with a physiologic glue, referred to as “fibrin glue”, prior to application of the growth factor intramyocardially. Fibrin glue typically comprises fibrinogen and thrombin, which react to form a fibrin matrix (see e.g. U.S. Pat. Nos. 4,377,572 and 4,642,120, WO 92/09301, and European Patent No. 0,068,047, which describes an anhydrous powder that is derived from an enriched plasma fraction that contains fibrinogen, a fibrinolysis inhibitor, and thrombin or prothrombin; the disclosures of which are incorporated herein by reference).

Fibrin glue may be purchased from IMMUNO AG or BEHRINGWERKE AG (Germany), as a two-component system comprising a fibrinogen component (trade name, “Tissucoll”) and a thrombin component (see U.S. Pat. No. owned by IMMUNO AG). The two components are mixed under sterile conditions at 37° C. in a calcium chloride solution in the presence of a protease inhibitor, immediately prior to combining with the angiogenic growth factor and application to the patient. However, while fibrin glue is contemplated in accordance with a preferred mode of practicing the present invention, other “glues” or matrix-forming compositions may also be used in accordance with the present disclosure.

An example of a variation of the fibrin matrix is disclosed in Australian Patent AU-A-75097/87, which describes a biological glue prepared from fibrinogen, factor XIII, a thrombin inhibitor, such as antithrombin III, prothrombin factors, calcium ions, and if necessary, a plasmin inhibitor. Other compounds which have been disclosed as useful in forming biological matrices include: collagen, elastin, Sepharose, gelatin and any other biodegradable material which forms a matrix (see e.g. WO 92/13565 to Hunziker; the disclosure of which is incorporated herein by reference).

The pharmaceutical composition, comprising the angiogenic growth factor and the physiologic glue is preferably prepared immediately prior to application. The growth factor may be prepared from a sterile concentrated stock solution, rehydrated from a lyophilized powder, or any other stable storage form recognized by those of ordinary skill in the biomedical field, and diluted in a sterile physiologic solution, preferably saline, to give a final volume of about 0.01 to 10 ml, preferably about 1 ml. This solution may optionally contain an anticoagulant, such as heparin, in a final concentration in a range of about 1 U per ml to about 1000 U per ml, or any other anticoagulant known in the art.

The solution containing the angiogenic factor is thoroughly mixed with the freshly prepared glue solution (about 0.01 to 10 ml; preferably about 1 ml) to yield a homogeneous solution containing the desired amounts of growth factor and glue in a volume suitable for injection intramyocardially. The volume of pharmaceutical composition injected per site should be in the range of about 0.02 to 5 ml, preferably not greater than about 2 ml. The pharmaceutical composition should be warmed to about 37° C. prior to application.

The following detailed description of the invention describes a procedure for producing and purifying human fibroblast growth factor-1 (hFGF-1), the preferred angiogenic growth factor in accordance with the present invention. Next, surgical methods for applying the pharmaceutical composition are described. Finally, working examples are presented for preparing, testing and using the pharmaceutical composition of the present invention.

A. Production and Purification of hFGF-1

### 1. Expression of hFGF-1 in E. coli

Standard recombinant deoxyribonucleic acid (DNA) techniques were used for the genetic engineering of apathogenic E. coli,. DeDuve, C. In: The Cell II, DeDuve, Heidelburg, Spektrum des Wiss: Verlages, pp. 366-367 (1986). Polyadenylated hFGF-1 messenger ribonucleic acid (mRNA) was extracted from human brain stem tissue using the method of de Ferra, F. et al. (Cell 13:721-727, 1985). The mRNA was then reverse transcribed using a DNA polymerase (reverse transcriptase) according to Jaye, M. et al. (Science 233:543-545, 1986) to obtain single-stranded complementary (c)DNA. The cDNA was then removed from a 1% agarose detection gel and purified by standard techniques.

The hFGF-1 cDNA was incorporated into a carrier plasmid pDS 25 as described by Forough, R. et al. (Biochim. Biophys. Acta 1090:393-398, 1991). Several clones (designated pDS 77, pDS 78, pDS 79 and pDS 76) were generated in this manner. These cDNA clones were introduced into expression vectors containing the trp-lac promoter. The resulting hFGF-1-α-pKK 233-2 expression vector was used to induce the synthesis of the hFGF-1 in E. coli.

The accurate fusion of the plasmids and vectors was checked by DNA sequencing at regular intervals. Amino acid sequencing was performed to confirm that the recombinant peptide produced by the E. coli was in fact hFGF-1.

The E. coli were cultured in a selective broth containing 50 μg/ml ampicillin, using 500 ml of culture medium for each bacterial strain. To prepare the nutrient broth, 10 g of Luria broth base was first dissolved in 500 ml of distilled water, and then 2.5 ml of a 0.5% NaCl solution and 12.5 mg of ampicillin per 500 ml were added. The pH of the broth was adjusted to 7.5 and then the broth was autoclaved at 121° C. and 1 bar for 30 min. After cooling the culture solution to room temperature, 10 ml were removed, pipetted into a sterile Erlenmeyer flask, and 20 μl genetically engineered E. coli was added. Then the nutrient broth inoculated with the bacteria was incubated overnight for 10-12 h in a rotary shaker at 37° C. at 150-200 rpm. A clear turbidity of the nutrient solution was visible to the naked eye, otherwise the broth was discarded, and a new E. coli culture was started.

After culturing overnight, 2 ml of culture medium containing the bacteria was removed from the sterile 490 ml culture flask and used as calibration solution for the photometer. The “overnight culture” (5 ml) of E. coli was then added at 37° C. to the remaining 488 ml of sterile broth, and the culturing was continued at 140-160 rpm for at least 2 h in a rotary shaker. The growth behavior of the bacteria was determined by sample collection at 20 min intervals and measurement of the optical density at 547 nm. When extinction values in a range of about 0.4 to about 0.7 for hFGF-1 were reached, the bacteria were immediately further processed. To induce the expression of the recombinant hFGF-1 proteins, 0.5 ml of a 1M isopropyl-p-D-thiogalactopyranoside (IPTG) solution was pipetted into the bacterial culture. Then the culture flask was left in the rotary shaker at 140-160 rpm and at a temperature of 37° C. for an additional 3 h. The tubes were then centrifuged at 9000 rpm in a GS-3 rotor at 4° C. for 10 min. The liquid supernatant produced was carefully decanted and discarded, or kept at 4° C. for the dot blot detection procedure.

The pellet, on the other hand, was checked for its consistency by shaking the tube slightly back and forth. No additional fluid should come out of the cell debris, otherwise the centrifugation process was repeated. It was only when the pellet was sufficiently solid that it was further used or frozen in 2 ml portions at −20° C. for further processing.

During the continuation of the pellet preparation, portions (about 4×2 ml) were each suspended in 1-2 ml Tris-EDTA-(TE) buffer and pipetted into 50 ml centrifugation tubes for freezing. A freshly centrifuged pellet could, in contrast, be left in its 50 ml centrifugation tube and resuspended with 4-8 ml of TE buffer. The subsequent steps then again resemble the methodological steps for processing thawed or fresh pellets.

For the preparation of the TE buffer, 0.37 g of EDTA and 1.58 g of Tris powder were mixed with 1000 ml of distilled water, and the pH was adjusted to 8 with 1 mM NaOH. A “lysis buffer” was used for the further resuspension of the bacterial components. For this purpose, 25 ml of TE buffer and 0.9 g of glucose were mixed in a 50 ml centrifugation tube and the pH was adjusted to 8 with 1 mM NaOH. The pellet was again suspended in “lysis buffer” by taking up the pellet into the pipette and expelling it from the pipette.

To extract the hFGF-1 from the bacterial bodies and bring them into solution, the pellet was reacted with 10 μg/ml of fresh hen's egg white lysozyme. For this purpose, the 50 ml centrifugation tube with the resuspended pellet was attached to a horizontal shaker, 250 pg of lysozyme was added, and the solution was incubated at 4° C. for 30 min with the enzyme.

### 2. Purification of hFGF-1

a) heparin-Sepharose chromatography—For subsequent purification of hFGF-1, a heparin-Sepharose gel was first prepared. All the work steps were carried out at a temperature of 4° C., and the TE buffer as well was at a temperature of 4° C. One stock gel solution was prepared for each factor, and it can be employed using a regeneration process for a total of 5 to 10 factor purification's. For the preparation of a fresh gel, 5 g of heparin-Sepharose powder was dissolved in 10 ml of TE buffer and washed with 1000 ml TE buffer using a suction filter. The solution was then filled into a 50 ml centrifugation tube and centrifuged at 4000 rpm and 0° C. for 5 min. The gel was then either processed directly or stored at 4° C. Any gel, which had already been used and which was stored at 4° C., could be reused after washing in a glass suction filter with 50-100 ml of TE buffer. Any gel residues in the centrifugation tube could be eluted with any desired amount of TE buffer and poured into the suction filter. The gel was then further washed with 1000 ml TE buffer, poured into a fresh 50 ml centrifugation tube and centrifuged at 4000 rpm and at 0° C. for 5 min. The further processing of the material was similar to that of freshly prepared heparin-Sepharose gel.

After a ready-for-use heparin-Sepharose gel was prepared, and expiration of the incubation time with the lysozyme, the resuspended E. coli pellet was divided into 5×5 ml fractions. The fractions were transported on ice to the sonicator, and each tube was sonicated for 20 sec at a rate of four pulses per second. The 5×5 ml pellets were then again combined and centrifuged for 10 min at 6000 rpm and 4° C. The supernatant was removed as completely as possible by careful pipetting, and the pellet was discarded. If needed, a 200 μl sample of the pellet and also of the supernatant were collected for the dot blot procedure and stored at 4° C.

The supernatant was then divided into 5×5 ml fractions, and each fraction was pipetted into sterile dialysis tubing (10 cm×1.5 cm, permeability up to MW=15,000 kD). The dialysis tubes were marked with their fraction numbers, placed vertically in a beaker filled with 500 ml TE buffer, and left for 12 h at 4° C. on a magnetic stirrer. 500 ml TE buffer was replaced every hour.

The supernatant obtained after centrifugation was mixed with the heparin-Sepharose gel directly in a batch-wise procedure. For this purpose, the 5×5 ml fractions of the dialyzed supernatant were carefully poured from the tubes into the 50 ml centrifugation tubes with the prepared gel, and moved with slight oscillation at 4° C. on a horizontal shaker, to avoid air bubbles, for at least 2 h.

As stock solution for the preparation of the column elution buffer, the Tris-EDTA (TE) buffer was used; it was always prepared in 20 liter portions, stored at 4° C. and used as pellet resuspension buffer and also as elution buffer for the purification of the chromatography columns. For cleaning the gel supernatant mixture out of the chromatography column, three additional buffer types were also used, which were prepared by adding different amounts of NaCl to the TE stock buffer. All the TE or TE-NaCl buffers were adjusted to pH 8. About 2 liters of pure TE buffer, was used for column cleaning. The hFGF-1 was finally eluted from the gel as the buffer molarities increased during elution of the column from 0.5 M to 0.65 M to 1.5 M.

After adsorption of the hFGF-1 to the heparin-Sepharose gel, purification of the growth factor was performed by column chromatography at 4° C. The elution buffer was maintained at a temperature of 4° C. This resulted in a considerable shortening of the cleaning time, because the gel-supernatant mixture was pre-rinsed before application onto the column with 1000 ml of TE buffer at 4° C. in a glass suction filter. The next step consisted of rinsing with 1000 ml of 0.5 M TE-NaCl buffer. The gel was then poured, without bubble formation, into the chromatography column.

Prior to elution of hFGF-1, the column was first washed with 500 ml of the 0.65 M TE-NaCl buffer at 4° C. A fraction collector set for 10 ml fractions was placed under the column. During elution, it was also possible to connect a peak meter which continuously monitors the optical densities and which indicates the order of exit of the factor fractions. As a rule, however, it was sufficient to collect fractions and to perform a specific detection of the hFGF-1 polypeptides later by sodium dodecyl sulfate polyacrylamide gel electrophoresis (SDS-PAGE). Elution of the hFGF-1 was accomplished by addition of 250 ml of 1.5 M TE-NaCl buffer. Care was taken that all 1.5 M buffer fractions which eluted from the column were collected. The 10 ml fractions were then stored at 4° C. for further processing. After the completion of the elution process, the column was closed and the eluted heparin-Sepharose gel was regenerated.

The fractions were dialyzed in 20 cm long dialysis tubes (permeability up to MW=15,000 kD), which had first been boiled for 10 min at 100° C. The dialysis was carried out against 1000 ml of TE buffer at 4° C. for 12 h, where the buffer was replaced every 3 h. The dialysis fractions were then stored at 4° C. for further processing.

b) SDS gel electrophoresis—The elution fractions were then further characterized using a positive protein detection in the Bio-Rad assay by 11.25% SDS-PAGE. The reference values used are the already mentioned molecular weights of hFGF-1 (MW=17,000 kD).

Two different gels are poured: the 11.25% acrylamide running gel was prepared by adding 12.5 ml of Rotiphorese stock solution to 1.26 ml of a 1% ammonium persulfate solution, 25 ml Tris (pH=8.8) and 10.24 ml of distilled water; the 1% ammonium persulfate solution was freshly prepared. After 5 min degassing of the gel preparation, 1 ml of a freshly prepared 10% SDS solution and 100 μl of tetraethylmethylenediamine (TEMED) were added, and the running gel was poured and immediately covered with distilled water.

A 3% stacking gel was prepared by mixing 1.5 ml of the Rotiphorese stock solution with 0.36 ml of 1% ammonium persulfate solution, 7.5 ml of Tris (pH=6.8) and 5.49 ml of distilled water. The solution was degassed for 5 min prior to addition of 150 μL of a 10% SDS solution and 60 μl of TEMED. The stacking gel was then poured.

The gel was then coated with upper buffer, which consisted of 3 g of glycine with 4.56 g of Tris and 1 g of the 10% SDS solution; after adjustment to pH 8.89, the total volume was brought up to 1000 ml with distilled water.

The hFGF-1 isolated by heparin-Sepharose column chromatography was tested for its purity by SDS-PAGE. 100 μl of each elution fraction and 10 μl of marker protein were applied to the gel. However, before application, the 100 μl of sample material was mixed with 100 μl of sample buffer. The sample buffer consisted of 5 ml Tris-HCl (1.25 M, pH 6.8), 2 g of 10% SDS solution, 5 ml of 2-mercaptoethanol, 5 ml of 87% glycerol, 28.4 ml of distilled water and 10 mg of bromophenol blue. 5 μl of a 10% SDS solution and 5 μl of mercaptoethanol were added to the 20 μl of the marker protein. The sample and the marker protein fractions were then heated for 10 min at 100° C. and pipetted onto the gel. A lower buffer (pH=7.47) was then filled into the bottom chamber of the electrophoresis apparatus; the lower buffer consisted of 250 ml of HCl (1 N), 37.85 g of Tris and 5 g of a 10% SDS solution, and the total volume was brought up to 5000 ml with distilled water. After a renewed addition of upper buffer into the upper chamber, voltage was applied at 300 V, 80 mA and 30 W.

After 3.5 h, the gel was placed into a staining solution, consisting of 4 g of Coomassie brilliant blue dissolved in 2000 ml of 50% methanol solution and 400 ml of 10% acetic acid, for 15 min and then the gel was filtered. The gel was then destained for 6-8 h in destaining solution, which consisted of 1080 ml of a 9% acetic acid solution with 600 ml of 5% methanol and 10 l of distilled water. The gels were stable in this destaining solution at room temperature for up to 7 days.

c) High-pressure liquid chromatography—A Vydac-C4 column (0.46×25 cm) was used for high-pressure liquid chromatography (HPLC). The HPLC procedure used was based on the procedure of Gospodarowicz, D. et al. (Proc. Natl. Acad. Sci. USA 81:6963-6967, 1984). Elution of the proteins was documented by monitoring the extinction values obtained by means of a connected photometer. Only those elution samples which had already been shown to contain hFGF-1 by protein determination and SDS-PAGE were used.

The chromatography column was equilibrated with 1000 ml of 0.1% trifluoroacetic acid solution and then packed with the selected elution fractions, a procedure during which the entire elution quantity was applied with a loading syringe at a loading rate of 1 ml/min in 10 ml fractions onto the column. The 0.1% elution buffer consisted of 1 ml of concentrated trifluoroacetic acid, which was mixed with 999 ml of concentrated acetonitrile. The hFGF-I was eluted from the HPLC column using a linear acetonitrile gradient of 26-36% acetonitrile. The elution was carried out at a flow rate of 1 ml/min for a total duration of 90 min.

An increase in the extinction values indicated exit of the protein fractions. The corresponding eluate was collected in 10 ml fractions and either stored at 4° C. for analysis by Western blot or lyophilized and stored below −20° C. In the final step, the HPLC column was thoroughly rinsed with 200 ml of trifluoroacetic acid buffer.

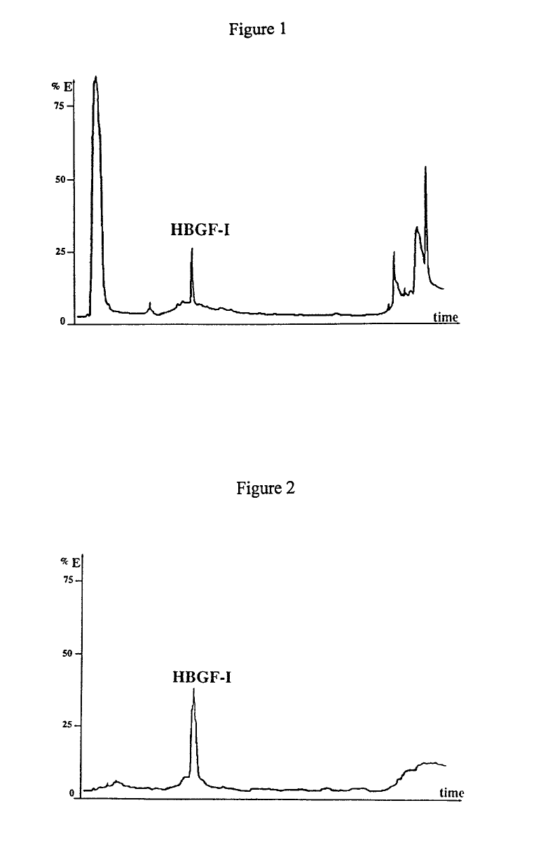

After separation, purification, and stabilization, we were able to isolate human hFGF-1 from 40 separate bacterial cultures and demonstrate its high degree of purity. FIG. 1 shows an HPLC profile of the growth factor after routine purification. The peak values at the beginning and end of the profile represent impurities that could be identified as E coli proteins. hFGF-1 could be further separated by fractionated collection, and the control HPLC (FIG. 2) merely shows the peak value of this fraction on an otherwise even baseline.

d) Western blot analysis—The biochemical isolation of the hFGF-1 factor was confirmed by the qualitative detection of the corresponding antigen by Western blot analysis. An anti-hFGF-1 IgG antibody was obtained from the Laboratory of Molecular Biology, American Red Cross, Rockville, USA. The Western blot specimens all consisted of HPLC elution fractions which corresponded to peak extinction values.

The same gels described for SDS-PAGE were used for the Western blots. After polymerization of the gels, the Western blot specimens were applied. For this purpose, a 20 μl sample was removed from each elution fraction, mixed with 100 μl of sample buffer, and heated for 10 min at 100° C. The sample buffer consisted of 5 ml of a 1.25 M Tris-HCl solution (pH=6.8), with 2 g SDS, 11.6 ml of 87% glycerol, 33.4 ml of distilled water and 10 mg of bromophenol blue. In addition, a marker solution was prepared as a control, in which the electrophoresis calibration powder from the calibration kit was dissolved in 80 μl of sample buffer and divided into 20 μl aliquots. To each aliquot of this marker solution, 5 μl of fresh 10% SDS solution and 5 μl of mercaptoethanol were added. This solution was heated for 10 min at 100° C.

After cooling, the sample and the markers were then pipetted directly onto the gel, using sample volumes of 120 μl and marker protein volumes of 30 μl. The gels were run at 100 V, 20 mA and 2 W. After 2 h, the gel plates were removed from the chamber and two graphite plates, wetted with distilled water, and 6 #3 filter papers, soaked in transfer buffer, were placed on the plates. The transfer buffer was obtained by the addition of 2.93 g of glycine, 5.81 g of TRIS, 0.375 g SDS and 200 ml methanol to distilled water, final volume of 1000 ml. The filter papers were covered with a nitrocellulose membrane with a pore size of 2 μm, and the gel was placed against the membrane. Six #3 filter papers soaked in sample buffer and an additional graphite plate were placed on top of the gel. Blotting was accomplished by application of 10 V, 150 mA and 5 W for 2 h.

The nitrocellulose membrane was placed into a dye dish with 200 ml of Ponceau red for 3 min and rinsed with distilled water until clear color bands were visible in the marker protein lanes. These bands were cut into individual strips. The marker bands were then placed into a dye dish with 200 ml of amido black for 8 min, and then in 200 ml of destaining solution. The sample strips, on the other hand, were saturated with 5% milk powder in a second dye dish and left for at least 30 min on a horizontal shaker with slight movement. The strips were then carefully transferred into a new dye dish, and again 15 ml of the 5% milk powder was applied by pipetting.

The anti-hFGF-1 antibodies were diluted in a BSA buffer solution before application to the nitrocellulose membrane. The BSA buffer was prepared by dissolving 1.58 g of Tris-HCl (10 mM) and 8.75 g NaCl (15 mM) in 1000 ml of distilled water, and 30 ml of this solution was mixed with 0.3 ml of concentrated BSA stock solution, and then the pH was adjusted to 8. A 1:100 dilution of the primary anti hFGF-1 antibody was prepared with this BSA buffer. The primary antibody was then applied in 1 μl portions onto the nitrocellulose membrane. The membrane was left for 1 h on the horizontal shaker with slight movement and then stored at 4° C. overnight. The membrane was washed three times for 10 min in 0.9% NaCl.

The second antibody, a 2% peroxidase-coupled anti-rabbit-anti-IgG antibody, was also diluted with BSA buffer, where 1 μl of the antibody was mixed with 74 μl of BSA buffer (1:75 dilution). The secondary antibody (10 μl/color band) was then applied to the sample strips and left for 30 min at room temperature on the rotary shaker.

The strips were washed again three times for 10 min in 0.9% NaCl and dyed with a carbazole solution, which consisted of 2 ml of a carbazole stock solution with 50 ml Na acetate buffer (pH 5) and 25 μl of a 30% hydrogen peroxide solution. As soon as the reaction bands become dyed, the strips were removed and thoroughly rinsed with distilled water. The colored bands were evaluated based on the migration speeds of the elution samples in comparison to the marker protein. In this process, the reaction strength of the sample materials with the anti-hFGF-1 antibodies, or the peroxidase-coupled anti-rabbit-anti-IgG antibodies, were taken into account. In this manner, it was possible to achieve specific detection of hFGF-1 using Western blot analysis.

B. Application of the Pharmaceutical Composition: Surgical Methods

The patient was placed in a supine position on the operating table and prepared and draped for a standard anterior left-sided thoracotomy incision. The left hemithorax was slightly elevated. Routine general anesthesia was induced via endotracheal intubation; the routine monitoring for open heart procedures including central venous and arterial lines was established. A left-sided curved transverse skin incision of six to eight centimeters was performed over the 5th rib anteriorly. Following minimal muscle dissection, the costal cartilage was divided at its junction with the end of the rib, and the left pleural space was entered. The pericardium was opened, and traction sutures were placed to retract the heart forward and to obtain stability of the operative field. The traction sutures also prevented the insufflated left lung from obscuring the surgeon's view.

The coronary artery system and its branches were explored and the lesions to be treated were identified. The described technique allowed access to the anterior (LAD), lateral (Cx) and apical portions of the heart, and the inferior diaphragmatic surface (RCA) as well if necessary. A β-blocker, such as Esmolol, was administered intravenously by the anesthesiologist, and the heart rate was reduced to about 20-60 beats per minute, preferably about 40-60 beats per minute.

The angiogenic pharmaceutical composition was prepared as detailed above, immediately prior to application. A suitable volume containing the desired dose of angiogenic growth factor was taken up in a syringe and administered intramyocardially using a standard 20 gauge needle into the target region of the underperfused myocardium. A maximum of three injections were generally performed, one each for the LAD, Cx, and RCA vascular beds. Each injection had to be performed in strong connection to the course of the native stenosed coronary artery. After completing the injection(s) and ascertaining that there was no bleeding, the pericardium was left open, a chest tube was inserted into the left pleural space, and the surgical incision was closed in the usual manner without pericostal sutures. The skin incision was closed using running reabsorbable suture material. The chest tube was required for about 12 to 24 hours. The patient was extubated in the operating room and postoperatively monitored in the usual manner for 24 hours. The average hospitalization was three days.

It is anticipated that catheter-based techniques may also be used for delivery of the angiogenic composition to the sites of stenosis. Indeed, catheters having steering means and application actuators are presently being used for ablation procedures in the atrial chambers and the delivery of local-acting pharmaceuticals and radiation doses at or near sites of vessel stenosis. Thus, percutaneous intraluminal catheter-based delivery means are also encompassed within the present disclosure.

C. Examples of Preparing, Testing and Using the Pharmaceutical Composition

### 1. Preparation of Fibrin Glue

In order to enhance the affinity of the growth factor for the myocardium, the growth factor was mixed with a physiologic glue referred to as “fibrin glue” prior to introduction in situ. A two-component human fibrin glue system was purchased from IMMUNO AG, Heidelberg, Germany). The fibrin glue was prepared using a “Fibrinotherm” temperature-controlled apparatus under sterile conditions.

First the apparatus was set to an operating temperature of 37° C. At the same time, the fibrinogen component (“Tissucoll”) was slowly thawed to room temperature. Then 19.6-26.5 mg of bovine thrombin-S and 3 ml of an aprotinin calcium chloride solution (with 3000 units kallidinogenase inactivator and 5.88 mg calcium chloride) were heated to a temperature of 37° C. The thrombin-S was combined with the aprotinin calcium chloride solution by pipetting under sterile conditions, mixed for 10 min with the magnetic stirrer and let stand at 37° C. The thawed “Tissucoll” material was dissolved in the calcium chloride solution, thoroughly mixed, and returned to the heating apparatus. Before use, all the solutions were completely homogeneous. In addition, care was taken that the solutions were stored at 37° C. until the time they were used.

### 2. Mixture of Growth Factor and Fibrin Glue

hFGF-1 (40 μg) was dissolved in 360 μl PBS-CMF (dilution 1:10), mixed, divided into 40×10 μl aliquots, and stored at −20° C. The quantity of growth factor used for one implantation was 10 μg (or 0.01 mg) per kg body weight of the diluted pure substance for each application site. Addition of the growth factor to the fibrin glue was carried out immediately prior to application, in the operating room. Since 1 ml of thrombin-S and 1 ml of “Tissucoll” were needed for each fibrin glue application, a 2 ml glue amount per implantation was used. After the stock solution of thrombin-S was prepared and ready at 37° C. in the “Fibrinotherm,” 1 ml was removed, mixed in a sterile tube with 10 μl of hFGF-1 and the combined quantity was then taken up into the application syringe. At the same time, 1 ml of “Tissucoll” stock solution was taken up into a second sterile syringe. Both the syringes containing the thrombin-S and the “Tissucoll” were stored at 37° C. until combined and administered to the patient.

### 3. In Vitro Studies

In in vitro experiments, we demonstrated the proliferative and mitogenic effects of the growth factor on human saphenous vein endothelial cells. Endothelial cell cultures with added growth factor induced a confluent monolayer after only 5 to 9 days, whereas the monolayer was not complete before 7 to 11 days in the control group (data not shown). In addition, to determining the total cell count with a cell counter, we also confirmed this result by analyzing the rate of DNA synthesis by measuring the incorporation of ³H-thymidine into the endothelial cell nuclei using the methods of Klagsbrun and Shing. The cell proliferative potency of hFGF-1 could be further intensified by adding heparin, a glycosaminoglycan protecting the growth factor from inactivation by cellular enzymes and from inactivation by cellular enzymes and from heat and chemical denaturation.

### 4. Chorioallantoic Membrane Assay

This established method, which provides a direct demonstration of the effect of growth factors on living tissue, was used to investigate the angiogenic effect of HFGF-1. The growth of the allantoic systems can be directly observed by light microscopy. After incubation of 20 fertilized hen eggs for 13 days, the growth factor was applied to the membrane and covered with tissue culture coverslips. Four days later, the membrane was examined under the light microscope and directly compared with controls untreated with hFGF-1 or treated with heat-denatured hFGF-1 (70° C. for 3 minutes).

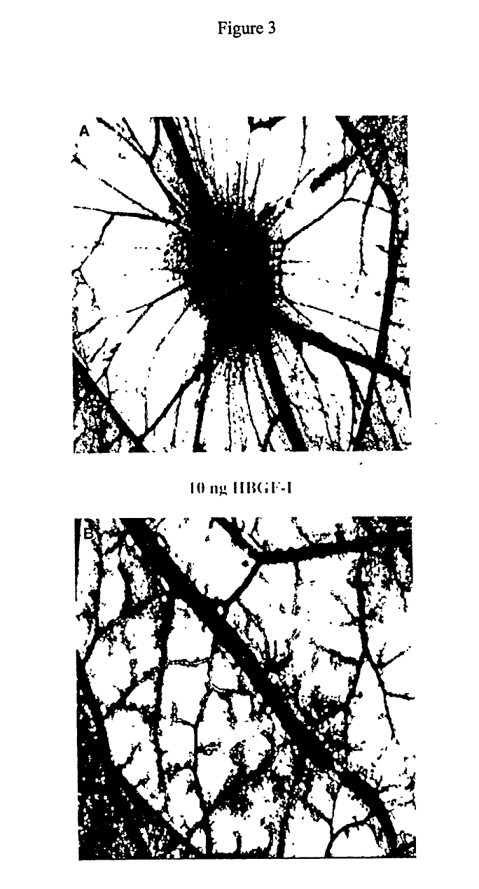

The angiogenic potency of hFGF-1 was demonstrated in vivo using the chorioallantoic membrane assay. As early as 4 days after application of the factor, the vascular structures of the membrane was completely altered. Emanating radially from the site of application, an unequivocal growth of new vessels from the original host vessels had grown out into the periphery (FIG. 3A). These structures were completely absent from the control group, and a normally developed reticular vascular pattern could be discerned (FIG. 3B). HBGF-I denotes hFGF-1.

### 5. Exclusion of the Pyrogenicity of hFGF-1

Varying concentrations of hFGF-1 (0.01, 0.5, or 1.0 mg/kg body weight) were injected subcutaneously, intramuscularly, or intravenously into 27 New Zealand White rabbits, the solvent alone being used for an additional 13 controls. Thereafter, the rectal temperature was taken every half hour for 3 hours, hourly for the rest of the day, and every 8 hours for 12 days. A daily white cell count was also repeated for 12 days. In addition to this, the erythrocyte sedimentation rate and the C-reactive protein values were determined on the 3rd, 6th, 9th, and 12th days after the injection.

Pyrogenic effects of the human growth factor produced in this way were definitively ruled out in the animal model. There was no significant rise of body temperature when checked at short intervals and no trace of an inflammatory reaction in comparison with the control group (n=13) in any of the 27 test animals during the period of observation. This result was independent of the concentration and the route of administration (intravenous, subcutaneous, or intramuscular) of the factor.

### 6. Exclusion of Tumor Stimulation by hFGF-1

To rule out the oncoproliferative effect of the growth factor, we did stimulation tests on human tumor cell lines. We investigated the following tumors by means of ³H-thymidine assays: pleomorphocellular sarcoma, hypernephroma, melanoma and small-cell lung cancer. The initial number of cells was 500 cells per well in 96-well plates. The tumor cell cultures were stimulated with different factor concentrations (10 and 100 ng HFGF-1). The total duration of stimulation was 24 hours.

In addition, human tumor cell lines were implanted in animal experiments for further preclusion of tumorigenicity. An initial dose of 3×10⁶ cells were implanted subcutaneously in a total of 80 nude mice. For this purpose, the tumor cells were taken up in 0.1 ml culture medium and injected subcutaneously into the right abdomen in the nude mice. The test animals were divided into four groups per tumor cell line: group 1 (n=20) received only tumor cells, group 2 (n=20) received tumor cells and systemic hFGF-1, group 3 (n=20) received a suspension of the tumor cells and growth factor, and group 4 (n=20) received only growth factor. Blood was taken from the animals and their live weight determined at 4-day intervals. After a test duration of 12 weeks, the tumors were explanted, their size and weight determined, and they were afterwards worked up histologically.

In the stimulation test carried out on various human tumor cell lines, possible tumor-stimulating effects of hFGF-1 were ruled out. An increased rate of DNA synthesis compared to the respective controls was not seen in any of the tumor cell lines. Moreover, hFGF-1 also failed to increase tumor cell growth in nude mice. Likewise, neither histological changes in tumors nor changes in the levels of tumor specific polypeptides were seen in animals treated with the growth factor. It is postulated that FGF receptor down-regulation following exposure to hFGF-1 resulted in a decreased ability of the growth factor to stimulate tumor cell growth. Thus, hFGF-1 treatment was not associated with any tumorigenic activity.

### 7. Angiogenic Potency of hFGF-1 in Animal Experiments

Supplementary to our earlier experiments, the effect of hFGF-1 was also investigated in the ischemic hearts of inbred Lewis rats (a total of 275 animals, including 125 controls treated with heat-denatured hFGF-1, 70° C. for 3 minutes). The pericardium was opened via the abdominal wall and diaphragm, and two titanium clips were inserted at the apex of the left ventricle to induce myocardial ischemia. Growth factor (mean concentration of 10 μg) was then injected locally into the site. The coronary vessel system was imaged by aortic root angiography after 12 weeks and, finally, a specimen from the same myocardial region was evaluated histologically.

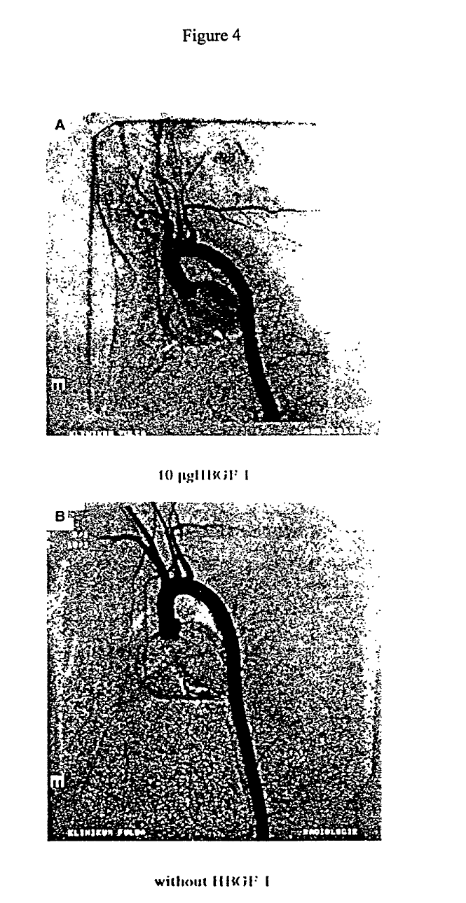

Proof of induced neoangiogenesis was found in the ischemic rat heart. In the test animals, in which myocardial ischemia had previously been induced with titanium clips and growth factor had subsequently been injected into the myocardium, a manifest accumulation of contrast medium was shown by aortic angiography at the site of the hFGF-1 injection 12 weeks later (FIG. 4A), whereas such an accumulation of contrast medium did not appear in any of the control animals (FIG. 4B). Histological examination of the myocardium revealed a threefold increase in the capillary density per square millimeter around the site of the hFGF-1 injection. HBGF-I denotes hFGF-1.

### 8. Clinical Use of hFGF-1 in Patients with CHD

This study was approved by the Medical Research Commission at the Phillips University of Marburg on Aug. 10, 1993 (No. 47.93). Twenty patients without any history of infarction or cardiac surgery (14 men and 6 women; minimum age, 50 years) were subjected to an elective bypass operation for multivessel coronary heart disease. The growth factor was applied directly during the operation. As a control group, 20 patients who underwent the same procedure were given heat-denatured hFGF-1 (70° C. for 3 minutes). The choice of treatment was completely random, the names being placed in sealed envelopes and selected in a blinded manner.

The details, nature, and aims of this procedure were explained beforehand to every patient who underwent the operation. In all cases, their fully informed consent was received. Both groups of patients were closely comparable with regard to clinical symptoms, accompanying disorders, cardiovascular risk factors, ventricular function, sex, and age. A comparable coronary morphology was found in both groups.

All patients had a further stenosis in the distal third of the LAD or at the origin of one of its branches in addition to a severe proximal stenosis. The mean ejection fraction of the left ventricle for all patients was 50%. The operative procedure for coronary revascularization with autologous grafts (an average per patient of 2 to 3 venous bypasses and 1 from the left IMA) was routinely performed. hFGF-1 (mean concentration, 0.01 mg/kg body weight) was injected into the myocardium distal to the IMA/LAD anastomosis and close to the LAD, during the maintenance of the extracorporeal circulation and after completion of the distal anastomosis. In the control group, heat-denatured hFGF-1 was substituted for active hFGF-1. After 12 weeks, the IMA bypasses of all the patients were imaged selectively by transfemoral, intra-arterial, and digital subtraction angiography.

Angiograms obtained in this way were evaluated by means of EDP-assisted digital gray-value analysis, a universally recognized and well-established technique for demonstrating capillary neoangiogenesis. Sites of interest both with and without hFGF-1 (meaning heat-denatured hFGF-1) were selected in the vessels filled with contrast medium and in regions of the myocardium distal to the IMA/LAD anastomosis. One hundred pixels were selected from each site of interest and analyzed digitally. Complete blackening of the x-ray films was rated with a gray value of 150, and areas without blackening of the film were allotted a zero value. During the first 5 postoperative days, separate laboratory checks in addition to the routine postoperative follow-up procedures were made twice daily, and the temperature checked three times a day.

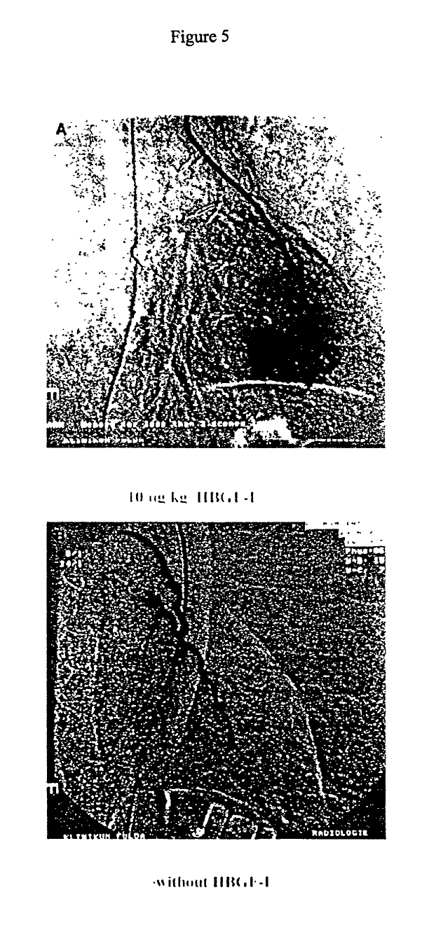

When the growth factor hFGF-1 was used clinically for the first time on the human heart, neoangiogenesis together with the development of a normal vascular appearance could be demonstrated angiographically. Selective imaging of the IMA bypasses by intra-arterial digital subtraction angiography confirmed the following result in all 20 patients: at the site of injection and in the distal areas supplied by the LAD, a pronounced accumulation of contrast medium extended peripherally around the artery for ≈3 to 4 cm, distal to the IMA/LAD anastomosis (FIG. 5A). HBGF-I denotes hFGF-1. In the control angiograms of patients to whom only heat-denatured hFGF-1 had been given, the IMA/LAD anastomosis was also recognizable, but the accumulation of contrast medium described above was absent (FIG. 5B). The angiograms of both the treated and control groups were recorded at a rate of four images per second, and these show comparable distances between the beginning of the injection and visualization of the medium.

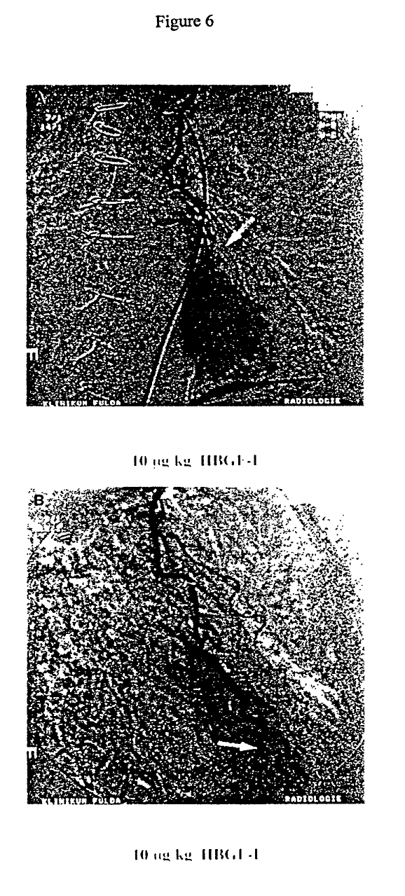

At the site of injection of the hFGF-1, a capillary network could be seen sprouting out from the coronary artery into the myocardium. This enabled retrograde imaging of a stenosed diagonal branch to be performed (FIG. 6A). such “neocapillary vessels” can also provide a collateral circulation around additional distal stenoses of the LAD (FIG. 6B) and bring about retrograde filling of a short segment of the artery distal to the stenosis. In none of the angiograms of the treated patients taken 12 weeks after the operation were any new stenoses of the LAD detectable.

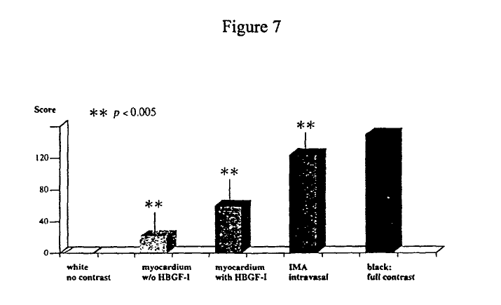

The results of EDP-assisted digital gray value analysis for quantification of the neoangiogenesis (FIG. 7) gave a mean gray value of 124 for the vessels. The control myocardium reached a gray value of only 20, and that of the myocardium injected with hFGF-1 gave a value of 59 (FIG. 7).

Importantly, the angiographic evidence of neovascularization was supported by enhanced ejection fractions in patients receiving hFGF-1, three years after surgery. The improvement in the blood supply, suggested post-operatively by angiography, was confirmed by results showing enhanced ejection fraction. Suprisingly, the improved vacularization as evidenced by enhanced ejection fraction was evident by echocardiographic follow up three years after the procedure. Indeed, the general ejection fraction in the study group improved from 50.3% before the operation to 63.8% after three years, whereas the the control group increased from 51.5% to only 59.4% within the same time period. These ejection fraction data were not predicted by the earlier animal studies and provide the first demonstration that neoangiogenesis in human myocardium is associated with an enhanced index of clinical function. Similarly, patients improved from NYHA III classification before the operation to NYHA I-II three years post-op. The marked improvement in cardiac function three years after growth factor therapy was suprising in view of the frequent incidence of restenosis in such patients.

On the basis of these in vitro and in vivo experiments, the efficacy of hFGF-1 for inducing neoangiogenesis in situ in the ischemic human heart and for treating CHD were established for the first time.

While a number of preferred embodiments of the invention and variations thereof have been described in detail, other modifications and methods of use will be readily apparent to those of skill in the art. Accordingly, it should be understood that various applications, modifications and substitutions may be made of equivalents without departing from the spirit of the invention or the scope of the claims.

What is claimed is:

1. A method for revascularizing a region of ischemic myocardium in a human heart which is underperfused as a result of at least one site of coronary artery stenosis, comprising the steps of:
preparing a pharmaceutical composition comprising fibroblast growth factor-1 (FGF-1) and a physiologic glue; and
injecting an amount of said pharmaceutical composition into the ischemic myocardium at or near the at least one site of coronary artery stenosis, said amount being sufficient to induce local neoangiogenesis.

2. The method of claim 1, wherein said FGF-1 is injected at a final concentration in a range of about 0.1 μg/kg body weight per site to about 10 mg/kg body weight per site.

3. The method of claim 1 wherein said FGF-1 is injected at a final concentration in a range of about 10 to 100 μg/kg body weight per site.

4. The method of claim 1, wherein said physiologic glue is fibrin glue.

5. The method of claim 1, wherein said FGF-1 and said physiologic glue are mixed immediately prior to application.

6. The method of claim 1, wherein said pharmaceutical composition further comprises an anticoagulant.

7. The method claim 6, wherein said anticoagulant is heparin.

8. The method of claim 7, wherein the heparin is applied at a final concentration in a range of about 1 U per ml to about 1000 U per ml.

9. The method of claim 1, wherein said injecting step further comprises:
making a thoracotomy incision;
identifying the at least one site of coronary artery stenosis;
administering a β-blocker to reduce the heart rate to a range of about 20-60 beats per minute; and
injecting the pharmaceutical composition intramyocardially at or near the at least one site of coronary artery stenosis.

10. The method of claim 9, wherein said thoracotomy incision further comprises an anterior left-sided incision; dissecting a region of costal cartilage over a 5th rib; and opening a left pleural space and a pericardium.

11. The method of claim 9, wherein the step of identifying the at least one site of coronary artery stenosis further comprises retracting the heart forward using traction sutures.
